(19) United States
(12) Patent Application Publication
(10) Pub. No.: US 2024/0325747 A1
(43) Pub. Date: Oct. 3, 2024

(54) COMPOSITIONS AND METHODS OF A CONCOMITANT THERAPY OF ALTERNATING ELECTRIC FIELDS AND N-CADHERIN INHIBITOR

(71) Applicant: NOVOCURE GMBH, Root D4 (CH)

(72) Inventors: ANAT KLEIN-GOLDBERG, Haifa (IL); ROM PAZ, Haifa (IL); TALI VOLOSHIN-SELA, Haifa (IL); LILACH AVIGDOR, Haifa (IL)

(21) Appl. No.: 18/621,825

(22) Filed: Mar. 29, 2024

Related U.S. Application Data
(60) Provisional application No. 63/493,389, filed on Mar. 31, 2023.
(60) Provisional application No. 63/593,160, filed on Oct. 25, 2023.

(51) Int. Cl. A61N 1/36; A61K 31/191; A61P 35/00; C07K 16/28; G01N 33/68

(52) U.S. Cl. CPC A61N 1/36002; A61K 31/191; A61P 35/00; C07K 16/2896; G01N 33/68

(57) ABSTRACT

Disclosed are methods of treating a subject in need thereof comprising applying an alternating electric field, at a frequency for a period of time, to a target site of the subject in need thereof; and administering a neural-cadherin (N-cadherin) inhibitor to the subject in need thereof. Disclosed are methods of preventing metastasis comprising applying an alternating electric field, at a frequency for a period of time, to a population of cells comprising one or more cancer cells; and contacting a N-cadherin inhibitor to the population of cells.

### CROSS-REFERENCE TO RELATED APPLICATIONS

This application claims the benefit of U.S. Provisional Patent Application No. 63/493,389, filed Mar. 31, 2023, and U.S. Provisional Patent Application No. 63/593,160, filed Oct. 25, 2023, each of which is incorporated by reference herein in its entirety.

### REFERENCE TO SEQUENCE LISTING

The Sequence Listing submitted Jun. 5, 2024 as a text file named “37983.0084U3.xml.” created on May 14, 2024, and having a size of 4,345 bytes is hereby incorporated by reference pursuant to 37 C.F.R. § 1.52 (e)(5).

### BACKGROUND

Neural (N)-cadherin is a calcium-dependent single-chain transmembrane glycoprotein that mediates homotypic and heterotypic cell-cell adhesion. As an important member of the cadherin family, N-cadherin plays an important role in the developmental and functional regulation of the nervous system, brain, heart, skeletal muscles, blood vessels and hematopoietic microenvironment. However, aberrant expression of N-cadherin has been found in many cancers, such as lung cancer, breast cancer, prostate cancer and squamous cell carcinoma. It is increasingly recognized that aberrant expression of N-cadherin is closely related to aspects of malignant tumor progression in humans, such as transformation, adhesion, apoptosis, angiogenesis, invasion and metastasis, indicating that N-cadherin can be a therapeutic target for tumor invasion and metastasis.

N-cadherin is intimately involved in the formation of blood vessels (a process known as angiogenesis) and the maintenance of their integrity.

N-cadherin antagonist LCRF-0006 inhibits neurite outgrowth and bone marrow endothelial cell (BMEC) adhesion in vitro. LCRF-0006 is also capable of disrupting BMEC monolayers, preventing endothelial tube formation in Matrigel, and disrupting mature endothelial tubes.

Inhibition of N-cadherin function destabilizes microvessels. For example, antibodies directed against N-cadherin disrupt endothelial cell-pericyte adhesive complexes and cause microvessels to hemorrhage.

Also, N-cadherin regulates the behavior of tumor cells in part due to the interaction with, and activation of, fibroblast growth factor receptor. FGFR has also been shown to be upregulated following TTFields expression.

Thus, preventing an increase or decreasing the expression of N-cadherin is beneficial in treating cancer.

### BRIEF SUMMARY

TTFields are shown herein to induce expression of N-cadherin. Due to the negative affects N-cadherin can have on cancer cells, the current invention is directed to concomitant therapy of TTFields and an N-cadherin inhibitor.

Disclosed are methods of treating a subject in need thereof comprising applying an alternating electric field, at a frequency for a period of time, to a target site of the subject in need thereof; and administering a neural-cadherin (N-cadherin) inhibitor to the subject in need thereof.

Disclosed are methods of preventing metastasis comprising applying an alternating electric field, at a frequency for a period of time, to a population of cells comprising one or more cancer cells; and contacting a N-cadherin inhibitor to the population of cells.

Disclosed are methods of reducing AKT phosphorylation in response to alternating electric fields comprising applying an alternating electric field, at a frequency for a period of time, to a population of cells comprising one or more fibroblasts; and contacting a calcium chelator to the population of cells.

Disclosed are methods of inhibiting the recruitment of the p85 subunit of PI3K to an N-cadherin complex in response to alternating electric fields comprising applying an alternating electric field, at a frequency for a period of time, to a population of cells; and contacting a calcium chelator to the population of cells.

Additional advantages of the disclosed methods and compositions will be set forth in part in the description which follows, and in part will be understood from the description, or may be learned by practice of the disclosed method and compositions. The advantages of the disclosed methods and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed methods and compositions and together with the description, serve to explain the principles of the disclosed methods and compositions.

FIG. 1 shows immunofluorescent imaging of non-small cell lung cancer carcinoma (NSCLC) cells (H1299 cells) treated with TTFields application for 72 hours.

FIG. 2 shows a schematic of an experimental design for testing N-cadherin accumulation in cell-cell junctions is dependent on calcium and N-cadherin activity.

FIG. 3 shows immunofluorescent imaging of NSCLC cells treated with TTFields application following 72 hours

FIG. 4 shows that AKT s473 phosphorylation is mediated by extra cellular Ca+ in H1299 cells.

FIG. 5 shows that AKT s473 phosphorylation is mediated by extra cellular Ca+ in A2780.

FIGS. 6A-6C show an example experiment of immunopreceipitation of N-cadherin following 3 days of TTFields treatment. (FIG. 6A) N-cadherin was immunoprecipitated from H1299 cell lysates with an anti-N-cadherin monoclonal antibody. The presence of the p85 subunit of PI3-kinase in anti-N-cadherin immunoprecipitation were detected by immunoblotting with PI3Kp85 antibody. Untreated cells included as control. (FIG. 6B) Western blot analysis of N-Cadherin, PI3K phosphorylated on P85 subunit, total PI3K, Akt phosphorylated on ser473 and total AKT levels in control and TTFields-treated cells. (FIG. 6C) Quantification of normalized relative levels of P-AKT compared to total AKT levels, normalized relative levels of P-PI3Kp85 compared to total PI3K levels and normalized N-cadherin levels in control and TTFields-treated cells. A value of 1 was given to the expression level of untreated cells. *p<0.05

FIGS. 7A-7C shows AKT Activation following TTFields is correlated with recruitment of the PI3Kp85 regulatory subunit to N-Cadherin. (FIG. 7A) N-cadherin was immunoprecipitated from A2780 cell lysates with an anti-N-cadherin monoclonal antibody. The presence of the p85 subunit of PI3-kinase in anti-N-cadherin immunoprecipitation were detected by immunoblotting with PI3Kp85 antibody. Untreated cells included as control. (FIG. 7B) Western blot analysis of N-Cadherin, PI3K phosphorylated on P85 subunit, total PI3K, Akt phosphorylated on ser473 and total AKT levels in control and TTFields-treated cells. (FIG. 7C) Quantification of normalized relative levels of P-AKT compared to total AKT levels, normalized relative levels of P-PI3Kp85 compared to total PI3K levels and normalized N-cadherin levels in control and TTFields-treated cells. A value of 1 was given to the expression level of untreated cells. *p<0.05

FIG. 8 shows AKT phosphorylation on s473 following TTFields is mediated by N-cadherin in H1299 cells.

FIG. 9 shows AKT phosphorylation on s473 following TTFields is mediated by N-cadherin in A2780 cells.

FIGS. 10A-10E show N-cadherin engagement functions upstream of AKT activation during long-term TTFields application. (FIG. 10A) H1299 cells were either left untreated or treated with TTFields (150 kHz) for 72 h. Left panel: Confocal fluorescence microscopy images of N-cadherin in control and TTFields-treated cells are shown. Blue, DAPI-stained DNA; Red, F-actin; Green, N-cadherin; Scale bars, 20 μm. Right panel: Quantification of N-cadherin expression, shown as mean±SEM. *p<0.05; unpaired t-test: N≥2. (FIGS. 10B, 10C) A2780 and H1299 cells were treated with TTFields (200 kHz and 150 kHz, respectively) for 72 h and then: left untreated (No EGTA), treated with 4 mM EGTA (EGTA), or treated with 4 mM EGTA subsequently replaced with serum-free calcium-containing medium (EGTA/Ca⁺²). (FIG. 10B) Representative phase-contrast images. Scale bars, 100 μm. (FIG. 10C) Upper panels: Samples were immunoblotted for AKT, pAKT (Ser473), and GAPDH. Lower panels: Densitometric analysis (arbitrary units normalized to the expression of the housekeeping protein GAPDH), is shown as mean±SEM. *p<0.05, and *** p<0.001; one-way ANOVA followed by Tukey's post hoc test: N≥2. (FIG. 10D) A2780 and H1299 cells were either left untreated or treated with TTFields (200 kHz and 150 kHz, respectively) for 72 h, followed by N-cadherin neutralization using an N-cadherin neutralizing antibody (N-cad nAb) directed against the extracellular domain of the protein. Upper panels: Samples immunoblotted for AKT, pAKT (Ser473), and GAPDH. Lower panels: Densitometric analysis is shown as the mean±SEM. *p<0.05. **p<0.01, and ***p<0.001; one-way ANOVA followed by Tukey's post hoc test; N≥2. (FIG. 10E) A2780 and H1299 cells were treated with TTFields (200 kHz and 150 kHz. respectively) for 72 h, followed by immunoprecipitation using an anti-N-cadherin antibody (α-N-cad). Samples were immunoblotted for N-cadherin and p85 regulatory subunit of PI3K. Non-specific IgG served as a negative control.

### DETAILED DESCRIPTION

The disclosed methods and compositions may be understood more readily by reference to the following detailed description of particular embodiments and the Example included therein and to the Figures and their previous and following description.

It is to be understood that the disclosed methods and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule. A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Headings are provided for convenience only and are not to be construed to limit the invention in any manner. Embodiments illustrated under any heading or in any portion of the disclosure may be combined with embodiments illustrated under the same or any other heading or other portion of the disclosure.

### A. Definitions

It is understood that the disclosed methods and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “an N-cadherin inhibitor” includes a plurality of such N-cadherin inhibitors, reference to “the N-cadherin inhibitor” is a reference to one or more N-cadherin inhibitors and equivalents thereof known to those skilled in the art, and so forth.

As used herein, a “target site” is a specific site or location within or present on a subject or patient. For example, a “target site” can refer to, but is not limited to a cell (e.g., a cancer cell or a cancer associated fibroblast), population of cells, organ, tissue, or a tumor, Thus, the phrase “target cell” can be used to refer to target site, wherein the target site is a cell. In some aspects, a “target cell” can be a cancer cell. In some aspects, organs that can be target sites include, but are not limited to, the ovaries or lungs. In some aspects, a cell or population of cells that can be a target site or a target cell include, but are not limited to, a cancer cell (e.g., a lung cancer cell). In some aspects, a “target site” can be a tumor target site.

A “tumor target site” is a site or location within or present on a subject or patient that comprises or is adjacent to one or more cancer cells, previously comprised one or more tumor cells, or is suspected of comprising one or more tumor cells. For example, a tumor target site can refer to a site or location within or present on a subject or patient that is prone to metastases. Additionally, a target site or tumor target site can refer to a site or location of a resection of a primary tumor within or present on a subject or patient. Additionally, a target site or tumor target site can refer to a site or location adjacent to a resection of a primary tumor within or present on a subject or patient.

As used herein, an “alternating electric field” or “alternating electric fields” refers to a very-low-intensity, directional, intermediate-frequency alternating electrical field delivered to a subject, a sample obtained from a subject or to a specific location within a subject or patient (e.g., a target site such as a cell). In some aspects, the alternating electrical field can be in a single direction or multiple directional, e.g., alternate directions across the target site. In some aspects, alternating electric fields can be delivered through two pairs of transducer arrays that generate perpendicular fields within the target site. For example, for the Optune™ system (an alternating electric fields delivery system) one pair of electrodes is located to the left and right (LR) of the target site, and the other pair of electrodes is located anterior and posterior (AP) to the target site. Cycling the field between these two directions (i.e., LR and AP) ensures that a maximal range of cell orientations is targeted.

As used herein, an “alternating electric field” applied to a tumor target site can be referred to as a “tumor treating field” or “TTField.” TTFields have been established as an anti-mitotic cancer treatment modality because they interfere with proper micro-tubule assembly during metaphase and eventually destroy the cells during telophase, cytokinesis, or subsequent interphase. TTFields target solid tumors and is described in U.S. Pat. No. 7,565,205, which is incorporated herein by reference in its entirety for its teaching of TTFields.

In-vivo and in-vitro studies show that the efficacy of TTFields therapy increases as the intensity of the electrical field increases. Therefore, optimizing array placement on a subject to increase the intensity in the target site or target cell is standard practice for the Optune system. Array placement optimization may be performed by “rule of thumb” (e.g., placing the arrays on the subject as close to the target site or target cell as possible), measurements describing the geometry of the patient's body, target site dimensions, and/or target site or cell location. Measurements used as input may be derived from imaging data. Imaging data is intended to include any type of visual data, such as for example, single-photon emission computed tomography (SPECT) image data, x-ray computed tomography (x-ray CT) data, magnetic resonance imaging (MRI) data, positron emission tomography (PET) data, data that can be captured by an optical instrument (e.g., a photographic camera, a charge-coupled device (CCD) camera, an infrared camera, etc.), and the like. In certain implementations, image data may include 3D data obtained from or generated by a 3D scanner (e.g., point cloud data). Optimization can rely on an understanding of how the electrical field distributes within the target site or target cell as a function of the positions of the array and, in some aspects, take account for variations in the electrical property distributions within the heads of different patients.

The term “subject” refers to the target of administration, e.g., an animal. Thus, the subject of the disclosed methods can be a vertebrate, such as a mammal. For example, the subject can be a human. The term does not denote a particular age or sex. “Subject” can be used interchangeably with “individual” or “patient.” For example, the subject of administration can mean the recipient of the alternating electrical field. For example, the subject of administration can be a subject with cancer, e.g., ovarian cancer or lung cancer.

By “treat” is meant to administer or apply a therapeutic, such as alternating electric fields and an N-cadherin inhibitor, to a subject, such as a human or other mammal (for example. an animal model), that has cancer or has an increased susceptibility for developing cancer, in order to prevent or delay a worsening of the effects of the disease or infection, or to partially or fully reverse the effects of cancer. For example, treating a subject having lung cancer can comprise delivering a therapeutic to a cell in the subject.

By “prevent” is meant to minimize or decrease the chance that a subject develops cancer.

As used herein, the terms “administering” and “administration” refer to any method of providing an N-cadherin inhibitor to a subject directly or indirectly to a target site. Such methods are well known to those skilled in the art and include, but are not limited to: oral administration, transdermal administration, administration by inhalation, nasal administration. topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat cancer. In further various aspects, a preparation can be administered prophylactically; that is, administered for prevention of cancer. In an aspect, the skilled person can determine an efficacious dose, an efficacious schedule, or an efficacious route of administration so as to treat a subject. In some aspects, administering comprises exposing or applying. Thus, in some aspects, exposing a target site or subject to alternating electrical fields or applying alternating electrical fields to a target site or subject means administering alternating electrical fields to the target site or subject.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Alternating Electric Fields

The methods disclosed herein comprise applying alternating electric fields. In some aspects, the alternating electric field used in the methods disclosed herein is a tumor-treating field. In some aspects, the alternating electric field can vary dependent on the type of cell or condition to which the alternating electric field is applied. In some aspects, the alternating electric field can be applied through one or more electrodes placed on the subject's body. In some aspects, there can be two or more pairs of electrodes. For example, arrays can be placed on the front/back and sides of a patient and can be used with the systems and methods disclosed herein. In some aspects, where two pairs of electrodes are used, the alternating electric field can alternate between the pairs of electrodes. For example, a first pair of electrodes can be placed on the front and back of the subject and a second pair of electrodes can be placed on either side of the subject, the alternating electric field can then be applied and can alternate between the front and back electrodes and then to the side to side electrodes.

In some aspects, the frequency of the alternating electric field is between 100 and 500 kHz. In some aspects, the frequency of the alternating electric field is between 50 kHz and 1 MHZ. The frequency of the alternating electric fields can also be, but is not limited to, between 50 and 500 kHz. between 100 and 500 kHz. between 25 kHz and 1 MHZ, between 50 and 190 kHz, between 25 and 190 kHz, between 150 and 300 kHz, between 180 and 220 kHz, or between 210 and 400 kHz. In some aspects, the frequency of the alternating electric fields can be 50 kHz, 100 kHz, 150 KHz, 200 KHz, 250 KHz. 300 KHz, 350 kHz, 400 KHz, 450 kHz, 500 KHz, or any frequency between. In some aspects, the frequency of the alternating electric field is from about 200 kHz to about 400 kHz, from about 250 kHz to about 350 kHz, and may be around 300 KHz.

In some aspects, the field strength of the alternating electric field can be between 0.5 and 4 V/cm RMS. In some aspects, the field strength of the alternating electric field can be between 1 and 4 V/cm RMS. In some aspects, different field strengths can be used (e.g., between 0.1 and 10 V/cm RMS). In some aspects, the field strength can be 1.75 V/cm RMS. In some embodiments the field strength is at least 1 V/cm RMS. In some aspects, the field strength can be 0.9 V/cm RMS. In other embodiments, combinations of field strengths are applied, for example combining two or more frequencies at the same time, and/or applying two or more frequencies at different times.

In some aspects, the alternating electric field can be applied for a variety of different intervals ranging from 0.5 hours to 72 hours. In some aspects, a different duration can be used (e.g., between 0.5 hours and 14 days). In some aspects, application of the alternating electric fields can be repeated periodically. For example, the alternating electric field can be applied every day for a two hour duration. For example, the alternating electric field can be applied for at least 4 hours per day, at least 8 hours per day, at least 12 hours per day, at least 16 hours per day, or at least 20 hours per day. In some aspects the alternating electric field can be applied for at least 4, 8, 12, 16, or 20 hours per day for at least 2 days. In some aspects the alternating electric field can be applied for at least 4, 8, 12, 16, or 20 hours per day for at least 3 days. In some aspects the alternating electric fields can be applied for at least 4, 8, 12, 16, or 20 hours per day for at least 7 days.

In some aspects, the consecutive exposure may last for at least 6 hours, at least 12 hours, at least 24 hours, at least 36 hours, at least 48 hours, or at least 72 hours or more.

In some aspects, the cumulative exposure may last for at least 42 hours, at least 84 hours, at least 168 hours, at least 250 hours, at least 400 hours, at least 500 hours, at least 750 hours, or more.

The disclosed methods comprise applying one or more alternating electric fields to a cell or to a subject. In some aspects, the alternating electric field is applied to a target site or tumor target site. When applying alternating electric fields to a cell, this can often refer to applying alternating electric fields to a subject comprising a cell. Thus, applying alternating electric fields to a target site of a subject results in applying alternating electric fields to a cell.

### C. Methods of Treating

Disclosed are methods of treating a subject in need thereof comprising applying an alternating electric field, at a frequency for a period of time, to a target site of the subject in need thereof; and administering a neural-cadherin (N-cadherin) inhibitor to the subject in need thereof.

In some aspects, the N-cadherin inhibitor is a calcium chelator. In some aspects, the calcium chelator is ethylene glycol-bis (β-aminoethyl ether)-N,N,N′,N′-tetraacetic acid (EGTA). Edetic acid, Citric acid, Edetate disodium anhydrous, or Edetate calcium disodium anhydrous.

In some aspects, the N-cadherin inhibitor is an N-cadherin antagonist. In some aspects, the N-cadherin antagonist is LCRF-0006, ADH-1 (CHAVC; SEQ ID NO:1), HAV-containing peptide (e.g. LRAHAVDNG; SEQ ID NO:2), Trp-containing peptide (e.g., SWTLYTPSGQSK; SEQ ID NO:3), Compound 15, HAV dimeric (e.g., CHAVDINGHAVDIC; SEQ ID NO:4), HAV-biomaterial (a linear peptide conjugated to a hydrogel, wherein the linear peptide can be any of those disclosed herein), or an N-cadherin antibody. In some aspects, the N-cadherin antibody is GC4, 2A9, or 1H7.

In some aspects, the subject in need thereof has cancer. In some aspects, the cancer can be, but is not limited to, ovarian cancer, non-small cell lung cancer, or breast cancer. In some aspects, the target site comprises cancer cells. In some aspects, the cancer cells can be, but are not limited to, ovarian cancer cells, non-small cell lung cancer cells, breast cancer cells, brain cancer cells, liver cancer cells, pancreatic cancer cells, prostate cancer cells.

In some aspects. the N-cadherin inhibitor reduces AKT phosphorylation. In some aspects, the AKT phosphorylation is the phosphorylation of Ser473.

In some aspects, the N-cadherin inhibitor reduces PI3K/p85 recruitment to N-cadherin.

In some aspects, the N-cadherin inhibitor reduces metastasis.

In some aspects, the alternating electric field is applied before, after, or simultaneously with administering the N-cadherin inhibitor. In some aspects, the step of applying the alternating electric fields begins at least one hour before an N-cadherin inhibitor. In some aspects, the step of applying the alternating electric fields begins at least 30 minutes before administering an N-cadherin inhibitor. In some aspects, applying the alternating electric fields simultaneously can mean applying within 5, 10, 15, 20, 25, 30, 35, 40, 45, 50, 55, or 60 minutes before or after administering an N-cadherin inhibitor. In some aspects, the alternating electric fields can be applied and the N-cadherin inhibitor administered at least 1, 2, 3, 4, 5, 6, 7, 8, 9, 10, 11, 12, 13, 14, 15, 16, 17, 18, 19, 20, 21, 22, 23, or 24 hours from each other.

In some aspects, the N-cadherin inhibitor is administered intratumorally, intracranially, intraventricularly, intrathecally, epidurally, intradurally, intravascularly, intravenously, intraarterially, intramuscularly, subcutaneously, intraperitoneally, orally, intranasally, topically, via intratumor injection, or via inhalation.

In some aspects, the frequency and/or field strength of the alternating electric field can be any of those described herein and can be applied in any of the manners described herein. In some aspects, the frequency of the alternating electric field is between 50 KHz and 1 MHZ. In some aspects, the frequency of the alternating electric field is about 150 or 250 kHz. In some aspects, the alternating electric field has a field strength of between 0.5 and 10 V/cm RMS. In some aspects, the alternating electric field has a field strength of about 0.9 V/cm RMS.

In some aspects, the methods of treating further comprise administering a cancer therapeutic. In some aspects, a cancer therapeutic can be any known cancer therapeutic, such as, but not limited to, a chemotherapeutic agent or anti-inflammatory agent.

In some aspects, after applying an alternating electric field and prior to administering an N-cadherin inhibitor, detecting an increase in N-cadherin expression in the subject or cell. Thus, in some aspects, the method comprises only administering an N-cadherin inhibitor to those subjects having an increase in N-cadherin after applying an alternating electric field.

In some aspects, administering an N-cadherin inhibitor is performed 1, 2, 3, 4, 5, 6, 7, 8, 9, or 10 days after applying an alternating electric field is performed. In some aspects, administering an N-cadherin inhibitor is performed simultaneously with applying an alternating electric field. In some aspects, administering an N-cadherin inhibitor is performed within hours, days, or weeks of applying an alternating electric field.

In some aspects, nanoparticles can be used in the disclosed methods. For example, in some aspects, chelators, such as N-cadherin inhibitors, can be carried by nanoparticles (e.g. encapsulated by nanoparticles) to provide specific chelation at the target site thus avoiding systemic influence of chelating calcium. In some aspects, the nanoparticles can be induced to release the N-cadherin inhibitor in the presence of alternating electric fields. For example, in some aspects, alternating electric fields can cause the nanoparticle to burst thus releasing the N-cadherin inhibitor. In some aspects, a nanoparticle can be a polymeric nanoparticle, liposome, micelle, metal nanoparticles. In some aspects, the nanoparticle comprises a target site-specific targeting moiety. In some aspects, the target site-specific targeting moiety can be a cancer cell-specific targeting moiety. In some aspects, the targeting moiety can direct, or target, the nanoparticle to a specific target site. The target site-specific targeting moiety can be a chemical, compound, peptide or nucleic acid. Examples of targeting moieties include, but are not limited to, molecules that recognize receptors on specific cell types.

### D. Method of Preventing Metastasis

Disclosed are methods of preventing metastasis comprising applying an alternating electric field, at a frequency for a period of time, to a population of cells comprising one or more cancer cells; and contacting an N-cadherin inhibitor to the population of cells.

In some aspects, the N-cadherin inhibitor is a calcium chelator. In some aspects, the calcium chelator is ethylene glycol-bis (β-aminoethyl ether)-N,N,N′,N′-tetraacetic acid (EGTA), Edetic acid, Citric acid, Edetate disodium anhydrous, or Edetate calcium disodium anhydrous.

In some aspects, the N-cadherin inhibitor is an N-cadherin antagonist. In some aspects, the N-cadherin antagonist is LCRF-0006, ADH-1 (CHAVC; SEQ ID NO:1), HAV-containing peptide (e.g. LRAHAVDNG; SEQ ID NO:2), Trp-containing peptide (e.g., SWTLYTPSGQSK; SEQ ID NO:3), Compound 15, HAV dimeric (e.g., CHAVDINGHAVDIC; SEQ ID NO:4), HAV-biomaterial (a linear peptide conjugated to a hydrogel, wherein the linear peptide can be any of those disclosed herein), or an N-cadherin antibody. In some aspects, the N-cadherin antibody is GC4, 2A9, or 1H7.

In some aspects, the cancer can be, but is not limited to, ovarian cancer, non-small cell lung cancer, breast cancer, brain cancer, liver cancer, pancreatic cancer, or prostate cancer. In some aspects, the target site comprises cancer cells. In some aspects, the cancer cells can be, but are not limited to, ovarian cancer cells, non-small cell lung cancer cells, brain cancer cells, liver cancer cells, pancreatic cancer cells, or prostate cancer cells.

In some aspects, the method is a method of preventing metastasis in a subject having cancer. For example, in some aspects, the population of cells is in vivo, thus, in some aspects, the population of cells is in a subject.

In some aspects, the population of cells comprises cancer cells. In some aspects, the target site comprises cancer cells. In some aspects, the cancer cells are ovarian cancer cells or non-small cell lung cancer cells.

In some aspects, the alternating electric field is applied before, after, or simultaneously with administering the N-cadherin inhibitor. In some aspects, the step of applying the alternating electric fields begins at least one hour before administering an N-cadherin inhibitor. In some aspects, the step of applying the alternating electric fields begins at least 30 minutes before an N-cadherin inhibitor. In some aspects, applying the alternating electric fields simultaneously can mean applying within 5, 10, 15, 20, 25, 30, 35, 40, 45, 50, 55, or 60 minutes before or after administering an N-cadherin inhibitor. In some aspects, the alternating electric fields can be applied and the N-cadherin inhibitor administered at least 1, 2, 3, 4, 5, 6, 7, 8, 9, 10, 11, 12, 13, 14, 15, 16, 17, 18, 19, 20, 21, 22, 23, or 24 hours from each other.

In some aspects, the N-cadherin inhibitor is administered intratumorally, intracranially, intraventricularly, intrathecally, epidurally, intradurally, intravascularly, intravenously, intraarterially, intramuscularly, subcutaneously, intraperitoneally, orally, intranasally, topically, via intratumor injection, or via inhalation.

In some aspects, the frequency and/or field strength of the alternating electric field can be any of those described herein and can be applied in any of the manners described herein. In some aspects, the frequency of the alternating electric field is between 50 kHz and 1 MHZ. In some aspects, the frequency of the alternating electric field is about 150 or 250 kHz. In some aspects, the alternating electric field has a field strength of between 0.5 and 10 V/cm RMS. In some aspects, the alternating electric field has a field strength of about 0.9 V/cm RMS.

In some aspects, the methods of treating further comprise administering a cancer therapeutic. In some aspects, a cancer therapeutic can be any known cancer therapeutic, such as, but not limited to, a chemotherapeutic agent or anti-inflammatory agent.

In some aspects, after applying an alternating electric field and prior to administering an N-cadherin inhibitor, detecting an increase in N-cadherin expression in the subject or cell. Thus, in some aspects, the method comprises only administering an N-cadherin inhibitor to those subjects having an increase in N-cadherin after applying an alternating electric field.

In some aspects, administering an N-cadherin inhibitor is performed 1, 2, 3, 4, 5, 6, 7, 8, 9, or 10 days after applying an alternating electric field is performed. In some aspects, administering an N-cadherin inhibitor is performed simultaneously with applying an alternating electric field. In some aspects, administering an N-cadherin inhibitor is performed within hours, days, or weeks of applying an alternating electric field.

In some aspects, nanoparticles can be used in the disclosed methods. For example, in some aspects, chelators, such as N-cadherin inhibtors, can be carried by nanoparticles (e.g. encapsulated by nanoparticles) to provide specific chelation at the target site thus avoiding systemic influence of chelating calcium. In some aspects, the nanoparticles can be induced to release the N-cadherin inhibitor in the presence of alternating electric fields. For example, in some aspects, alternating electric fields can cause the nanoparticle to burst thus releasing the N-cadherin inhibitor. In some aspects, a nanoparticle can be a polymeric nanoparticle, liposome, micelle, metal nanoparticles. In some aspects, the nanoparticle comprises a target site-specific targeting moiety. In some aspects, the target site-specific targeting moiety can be a cancer cell-specific targeting moiety. In some aspects, the targeting moiety can direct, or target, the nanoparticle to a specific target site. The target site-specific targeting moiety can be a chemical, compound, peptide or nucleic acid. Examples of targeting moieties include, but are not limited to, molecules that recognize receptors on specific cell types.

### E. Methods of Reducing AKT Phosphorylation

Disclosed are methods of reducing AKT phosphorylation in response to alternating electric fields comprising applying an alternating electric field, at a frequency for a period of time, to a population of cells comprising one or more fibroblasts: and contacting a calcium chelator to the population of cells. In some aspects, the calcium chelator is ethylene glycol-bis (β-aminoethyl ether)-N,N,N′,N′-tetraacetic acid (EGTA), Edetic acid, Citric acid, Edetate disodium anhydrous, or Edetate calcium disodium anhydrous.

In some aspects, N-cadherin is activated by calcium binding so calcium can act as an inhibitor of N-cadherin activity. In some aspects, besides a calcium chelator, other N-cadherin inhibitors can be used. Thus, disclosed are methods of reducing AKT phosphorylation in response to alternating electric fields comprising applying an alternating electric field, at a frequency for a period of time, to a population of cells comprising one or more fibroblasts; and contacting an N-cadherin inhibitor to the population of cells. In some aspects, an N-cadherin inhibitor can effect neurotransmission and cardiac signaling. In some aspects, the N-cadherin inhibitor is an N-cadherin antagonist. In some aspects, the N-cadherin antagonist is LCRF-0006, ADH-1 (CHAVC; SEQ ID NO:1), HAV-containing peptide (e.g. LRAHAVDNG; SEQ ID NO: 2), Trp-containing peptide (e.g., SWTLYTPSGQSK; SEQ ID NO:3), Compound 15, HAV dimeric (e.g., CHAVDINGHAVDIC; SEQ ID NO:4), HAV-biomaterial (a linear peptide conjugated to a hydrogel, wherein the linear peptide can be any of those disclosed herein), or an N-cadherin antibody. In some aspects, the N-cadherin antibody is GC4, 2A9, or 1H7.

In some aspects, the population of cells is in vivo, thus, in some aspects, the population of cells is in a subject. In some aspects, contacting a calcium chelator, or N-cadherin inhibitor, to the population of cells can include administering a calcium chelator, or N-cadherin inhibitor, to a subject comprising the population of cells.

In some aspects, the frequency and/or field strength of the alternating electric field can be any of those described herein and can be applied in any of the manners described herein. In some aspects, the frequency of the alternating electric field is between 50 kHz and 1 MHZ. In some aspects, the frequency of the alternating electric field is about 150 or 250 kHz. In some aspects, the alternating electric field has a field strength of between 0.5 and 10 V/cm RMS. In some aspects, the alternating electric field has a field strength of about 0.9 V/cm RMS.

### F. Methods of Inhibiting the Recruitment of p85

Disclosed are methods of inhibiting the recruitment of the p85 subunit of PI3K to an N-cadherin complex in response to alternating electric fields comprising applying an alternating electric field, at a frequency for a period of time, to a population of cells: and contacting a calcium chelator to the population of cells. In some aspects, the calcium chelator is ethylene glycol-bis (β-aminoethyl ether)-N,N,N′,N′-tetraacetic acid (EGTA), Edetic acid, Citric acid, Edetate disodium anhydrous, or Edetate calcium disodium anhydrous. In some aspects, the population of cells comprises one or more fibroblasts.

In some aspects, besides a calcium chelator, other N-cadherin inhibitors can be used. Thus, disclosed are methods of reducing AKT phosphorylation in response to alternating electric fields comprising applying an alternating electric field, at a frequency for a period of time, to a population of cells comprising one or more fibroblasts: and contacting an N-cadherin inhibitor to the population of cells. In some aspects, an N-cadherin inhibitor can effect neurotransmission and cardiac signaling. In some aspects, the N-cadherin inhibitor is an N-cadherin antagonist. In some aspects, the N-cadherin antagonist is LCRF-0006, ADH-1 (CHAVC; SEQ ID NO:1), HAV-containing peptide (e.g. LRAHAVDNG; SEQ ID NO:2), Trp-containing peptide (e.g., SWTLYTPSGQSK; SEQ ID NO:3), Compound 15. HAV dimeric (e.g., CHAVDINGHAVDIC; SEQ ID NO:4), HAV-biomaterial (a linear peptide conjugated to a hydrogel, wherein the linear peptide can be any of those disclosed herein), or an N-cadherin antibody. In some aspects, the N-cadherin antibody is GC4, 2A9, or 1H7.

In some aspects, the population of cells is in vivo, thus, in some aspects, the population of cells is in a subject. In some aspects, contacting a calcium chelator, or N-cadherin inhibitor, to the population of cells can include administering a calcium chelator, or N-cadherin inhibitor, to a subject comprising the population of cells.

In some aspects, the frequency and/or field strength of the alternating electric field can be any of those described herein and can be applied in any of the manners described herein. In some aspects, the frequency of the alternating electric field is between 50 KHz and 1 MHZ. In some aspects, the frequency of the alternating electric field is about 150 or 250 kHz. In some aspects, the alternating electric field has a field strength of between 0.5 and 10 V/cm RMS. In some aspects, the alternating electric field has a field strength of about 0.9 V/cm RMS.

### G. Compositions

Disclosed are compositions and formulations comprising one or more N-cadherin inhibitors. In some embodiments the formulation further includes a pharmaceutically acceptable carrier or diluent. For example, disclosed are pharmaceutical compositions, comprising an N-cadherin inhibitor and a pharmaceutically acceptable carrier. For example, disclosed are pharmaceutical compositions, comprising LCRF-0006, ADH-1, HAV-containing peptide, Trp-containing peptide, Compound 15, HAV dimeric, HAV-biomaterial, N-cadherin antibody, or H-SWTLYTPSGQSK-NH2 and a pharmaceutically acceptable carrier. Disclosed also are pharmaceutical compositions, comprising an N-cadherin inhibitor and a pharmaceutically acceptable diluent.

In some aspects, the N-cadherin inhibitor can be administered with a pharmaceutically acceptable carrier and/or diluent in any of the disclosed methods.

For example, the compositions described herein can comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidylcholine (DMPC), phosphate buffered saline or a multivesicular liposome. For example, PG: PC: Cholesterol: peptide or PC: peptide can be used as carriers in this invention. Other suitable pharmaceutically acceptable carriers and their formulations are described in Remington: The Science and Practice of Pharmacy (19th ed.) ed. A.R. Gennaro, Mack Publishing Company, Easton, PA 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), gels (including hydrogels), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. In the methods described herein, delivery of the disclosed compositions to cells can be via a variety of mechanisms. The pharmaceutical composition may be administered in a number of ways depending on whether local or systemic treatment is desired, and on the area to be treated.

In some aspects, the compositions comprise a nanoparticle carrying one or more N-cadherin inhibitors. In some aspects, a nanoparticle can be a polymeric nanoparticle, liposome, micelle, metal nanoparticles. In some aspects, the nanoparticle comprises a target site-specific targeting moiety. In some aspects, the target site-specific targeting moiety can be a cancer cell-specific targeting moiety. In some aspects, the targeting moiety can direct, or target, the nanoparticle to a specific target site. The target site-specific targeting moiety can be a chemical, compound, peptide or nucleic acid. Examples of targeting moieties include, but art not limited to, molecules that recognize receptors on specific cell types.

### 1. Delivery of Compositions

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid-or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, tri-alkyl and aryl amines and substituted ethanolamines.

### H. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more of N-cadherin inhibitors and one or more materials for delivering alternating electric fields, such as the Optune R system. For example disclosed are kits comprising one or more of an N-cadherin inhibitor and one or more materials for delivering alternating electric fields, such as the Optune R system. In some aspects, the kits can also include a cancer therapeutic.

### EMBODIMENTS

Embodiment 1: A method of treating a subject in need thereof comprising: applying an alternating electric field, at a frequency for a period of time, to a target site of the subject in need thereof; and administering a neural-cadherin (N-cadherin) inhibitor to the subject in need thereof.

Embodiment 2: The method of embodiment 1, wherein the N-cadherin inhibitor is a calcium chelator.

Embodiment 3: The method of embodiment 2, wherein the calcium chelator is EGTA, Edetic acid, Citric acid, Edetate disodium anhydrous, or Edetate calcium disodium anhydrous.

Embodiment 4: The method of embodiment 1, wherein the N-cadherin inhibitor is an N-cadherin antagonist.

Embodiment 5: The method of embodiment 4, wherein the N-cadherin antagonist is LCRF-0006, ADH-1, HAV-containing peptide, Trp-containing peptide, Compound 15, HAV dimeric, HAV-biomaterial, N-cadherin antibody, H-SWTLYTPSGQSK-NH2.

Embodiment 6: The method of embodiment 5, wherein the N-cadherin antibody is GC4, 2A9, or 1H7.

Embodiment 7: The method of any one of embodiments 1-6, wherein the subject has cancer.

Embodiment 8: The method of embodiment 7, wherein the cancer is ovarian cancer or non-small cell lung cancer.

Embodiment 9: The method of any one of embodiments 1-8, wherein the N-cadherin inhibitor reduces AKT phosphorylation.

Embodiment 10: The method of any one of embodiments 1-9, wherein the N-cadherin inhibitor reduces PI3K/p85 recruitment to N-cadherin.

Embodiment 11: The method of any one of embodiments 1-10, wherein the N-cadherin inhibitor reduces metastasis.

Embodiment 12: A method of reducing AKT phosphorylation in response to alternating electric fields comprising: a) applying an alternating electric field, at a frequency for a period of time, to a population of cells comprising one or more fibroblasts: and b) contacting a calcium chelator to the population of cells.

Embodiment 13: A method of inhibiting the recruitment of the p85 subunit of PI3K to an N-cadherin complex in response to alternating electric fields comprising: a) applying an alternating electric field, at a frequency for a period of time, to a population of cells; and b) contacting a calcium chelator to the population of cells.

Embodiment 14: The method of any one of embodiments 12-13, wherein the calcium chelator is EGTA.

Embodiment 15: A method of preventing metastasis in a subject having cancer comprising: a) applying an alternating electric field, at a frequency for a period of time, to a population of cells comprising one or more cancer cells; and b) contacting a N-cadherin inhibitor to the population of cells.

Embodiment 16: The method of embodiment 15, wherein the N-cadherin inhibitor is a calcium chelator.

Embodiment 17: The method of embodiment 16, wherein the calcium chelator is EGTA, Edetic acid, Citric acid, Edetate disodium anhydrous, or Edetate calcium disodium anhydrous.

Embodiment 18: The method of embodiment 15, wherein the N-cadherin inhibitor is an N-cadherin antagonist.

Embodiment 19: The method of embodiment 18, wherein the N-cadherin antagonist is LCRF-0006, ADH-1, HAV-containing peptide, Trp-containing peptide, Compound 15, HAV dimeric, HAV-biomaterial, N-cadherin antibody, H-SWTLYTPSGQSK-NH2.

Embodiment 20: The method of any of the preceding embodiments, wherein the alternating electric field is applied before, after, or simultaneously with administering the N-cadherin inhibitor.

Embodiment 21: The method of any of the preceding embodiments, wherein the N-cadherin inhibitor is administered intratumorally, intracranially, intraventricularly, intrathecally, epidurally, intradurally, intravascularly, intravenously, intraarterially, intramuscularly, subcutaneously, intraperitoneally, orally, intranasally, topically, via intratumor injection, or via inhalation.

Embodiment 22: The method of any one of embodiments 12-21, wherein the population of cells is in vivo.

Embodiment 23: The method of any one of embodiments 12-21, wherein the population of cells is in a subject.

Embodiment 24: The method of any of the preceding embodiments, wherein the frequency of the alternating electric field is between 50 kHz and 1 MHZ.

Embodiment 25: The method of any of the preceding embodiments, wherein the frequency of the alternating electric field is about 150 or 250 kHz.

Embodiment 26: The method of any of the preceding embodiments, wherein the alternating electric field has a field strength of between 0.5 and 10 V/cm RMS.

Embodiment 27: The method of any of the preceding embodiments, wherein the alternating electric field has a field strength of about 0.9 V/cm RMS.

Embodiment 28: The method of any of the preceding embodiments, further comprising administering a cancer therapeutic.

Embodiment 29: The method of any one of embodiments 1-28, wherein after step a) and prior to step b) detecting an increase in N-cadherin expression in the subject or cell.

Embodiment 30: The method any one of embodiments 1-29, wherein step b) is performed 1, 2, 3, 4, 5, 6, 7, 8, 9, or 10 days after step a) is performed.

Embodiment 31: The method of any one of embodiments 12-30, wherein the population of cells comprises cancer cells.

Embodiment 32: The method of any one of embodiments 1-30, wherein the target site comprises cancer cells.

Embodiment 33: The method of any one of embodiments 31 or 32, wherein the cancer cells are ovarian cancer cells or non-small cell lung cancer cells.

Embodiment 34: A composition comprising a neural-cadherin (N-cadherin) inhibitor for use in a method of treating a subject in need thereof, the method comprising: applying an alternating electric field to a target site of the subject in need thereof; and administering theneural-cadherin (N-cadherin) inhibitor to the subject in need thereof.

Embodiment 35: The composition of embodiment 34, wherein the subject has cancer, optionally wherein the cancer is ovarian cancer or non-small cell lung cancer.

Embodiment 36: The composition of any one of embodiments 34-35, wherein the N-cadherin inhibitor reduces AKT phosphorylation, reduces PI3K/p85 recruitment to N-cadherin, or reduces metastasis in the subject.

Embodiment 37: A composition comprising a calcium chelator for use in a method of reducing AKT phosphorylation or inhibiting the recruitment of the p85 subunit of PI3K to an N-cadherin complex in response to alternating electric fields, the method comprising: a) applying an alternating electric field to a population of cells comprising one or more fibroblasts; and b) contacting the calcium chelator to the population of cells.

Embodiment 38: A composition comprising a neural-cadherin (N-cadherin) inhibitor for use in a method of preventing metastasis in a subject having cancer, the method comprising: a) applying an alternating electric field to a population of cells comprising one or more cancer cells; and b) contacting the N-cadherin inhibitor to the population of cells.

Embodiment 39: The composition of any of embodiments 34-38, wherein the N-cadherin inhibitor is a calcium chelator or an N-cadherin antagonist.

Embodiment 40: The composition of embodiment 39, wherein the calcium chelator is EGTA, Edetic acid, Citric acid, Edetate disodium anhydrous, or Edetate calcium disodium anhydrous.

Embodiment 41: The composition of embodiment 39, wherein the N-cadherin antagonist is LCRF-0006, ADH-1, HAV-containing peptide, Trp-containing peptide, Compound 15, HAV dimeric, HAV-biomaterial, N-cadherin antibody, or H-SWTLYTPSGQSK-NH2.

Embodiment 42: The composition of any one of embodiments 34-41, wherein the alternating electric field is applied before, after, or simultaneously with administering the N-cadherin inhibitor.

Embodiment 43: The composition of any one of embodiments 34-42, wherein the alternating electric field is applied at a frequency between 50 KHz and 1 MHZ, optionally about 150 or 250 KHz.

Embodiment 44: The composition of any one of embodiments 34-43, wherein the alternating electric field has a field strength of between 0.5 and 10 V/cm RMS, optionally about 0.9 V/cm RMS.

Embodiment 45: The composition of any one of embodiments 34-44, further comprising administering a cancer therapeutic.

Embodiment 46: The composition of any one of embodiments 34-45, wherein after step a) and prior to step b) detecting an increase in N-cadherin expression in the subject or cell.

Embodiment 47: The composition of any one of embodiments 34-46, wherein step b) is performed 1, 2, 3, 4, 5, 6, 7, 8, 9, or 10 days after step a) is performed.

Embodiment 48: The composition of any one of embodiments 34-47, wherein the target site comprises cancer cells, optionally ovarian cancer cells or non-small cell lung cancer cells.

### EXAMPLES

### A. Example 1

Preclinical data by Novocure's team has shown that TTFields treatment, in NSCLC cells, increases expression of N-cadherin in a calcium-dependent manner compared to untreated cells, in vitro.

The most studied cyclic peptide is N-Ac-CHAVC-NH2 (designated ADH-1), which is capable of disrupting a wide variety of N-cadherin-mediated processes. Importantly, ADH-1 has been shown to inhibit angiogenesis, thus would improve TTFields treatment outcome. Systemic ADH-1, was reported to play dual function to both: (1) effect vascular permeability in the tumor microenvironment and (2) modulate tumor growth through activation of the AKT pathway.

### 1. Results

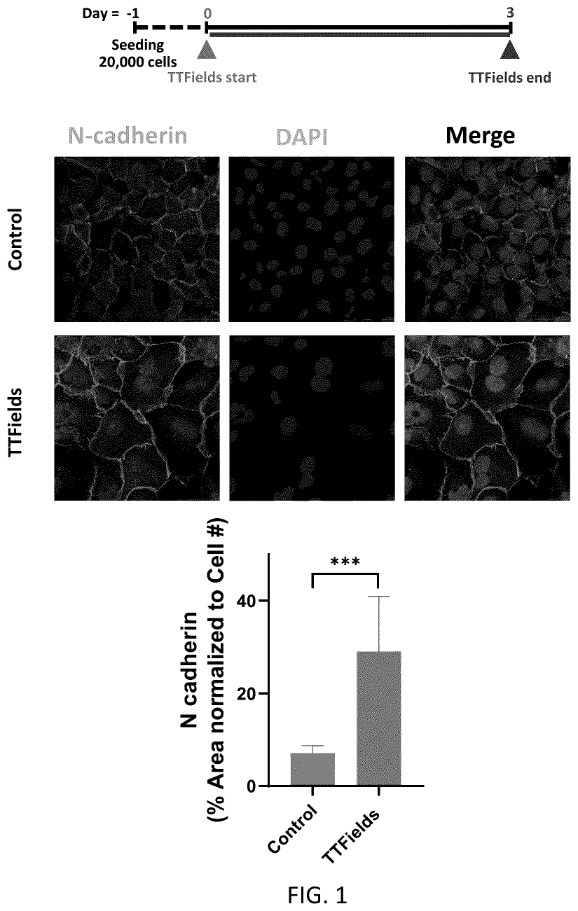

FIG. 1 shows that N-cadherin expression is enhanced following application of TTFields. Results indicated an increase in N-cadherin following TTFields compared to untreated cells.

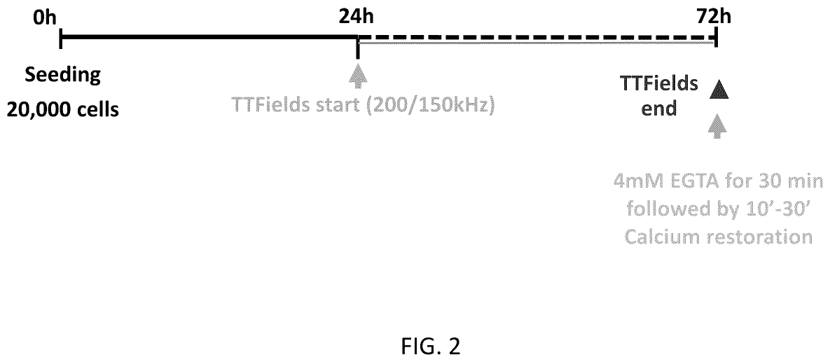

FIG. 2 shows a schematic of an experimental design for N-cadherin accumulation in cell-cell junctions is dependent on calcium and N-cadherin activity. In an example Ca²⁺ switch experiment. NSCLC cells were treated with TTFields application for 72 hours. Following 72 hours cells were left untreated or treated with 4 mM EGTA (calcium chelating agent) for 30 min. The EGTA-containing medium was then replaced with calcium containing medium for 30 min. The cells can be H1299 or A2780 cells.

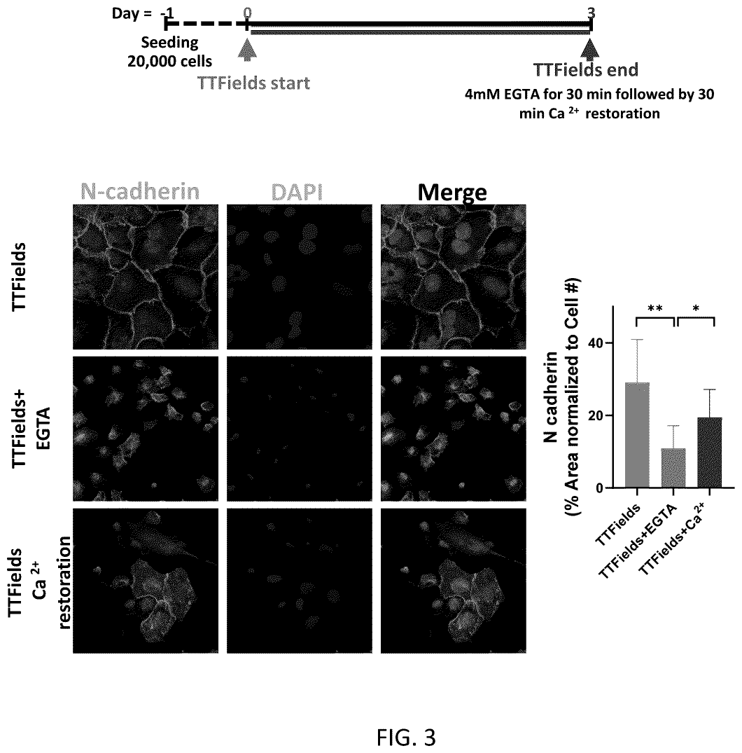

FIG. 3 shows N-cadherin expression is enhanced following TTFields and is Ca+ dependent. N-cadherin accumulation in cell-cell junctions is dependent on calcium and N-cadherin activity as described in FIG. 3. Results indicated that activation of N-cadherin following TTFields application is calcium dependent and following calcium restoration N-cadherin is reactive.

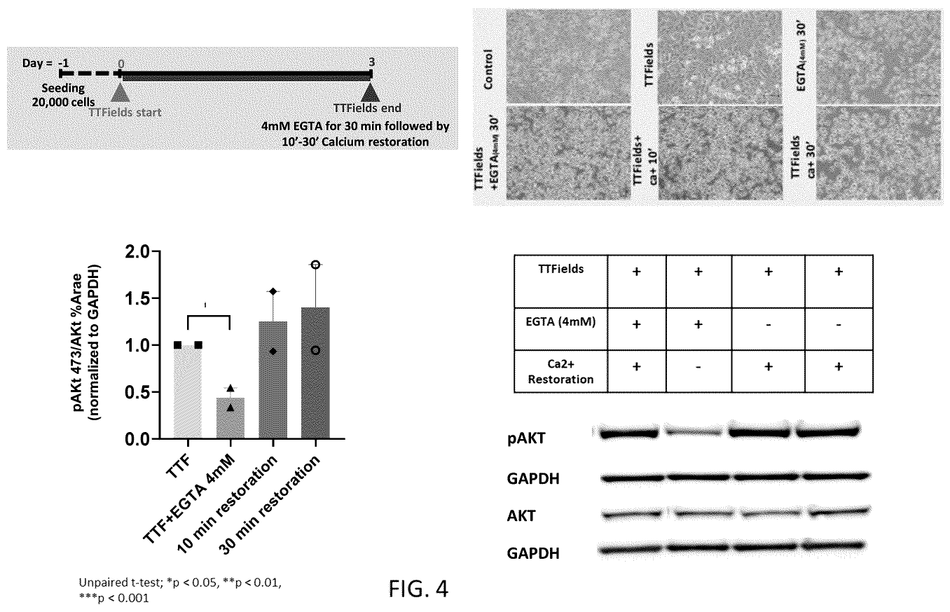

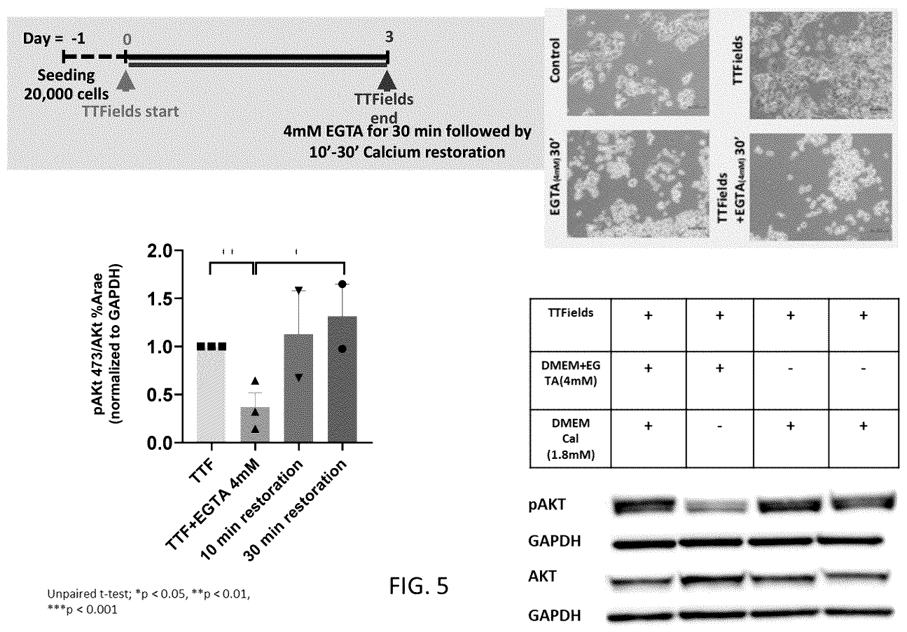

FIG. 4 and FIG. 5 show that AKT s473 phosphorylation is mediated by extra cellular Ca²⁺. NSCLC cells (FIGS. 4—H1299 cells) or ovarian cancer cells (FIGS. 5—A2780 cells) treated with TTFields for 72 hours were incubated with 4 mm EGTA to disrupt cell-cell contacts. Immunoblot analyses of phosphorylated-Akt on the Ser-473 amino acid. Cellular lysates were separated on a 15% SDS-PAGE, transferred onto nitrocellulose, and immunoblotted with antibodies to phospho-specific Akt on Ser-473. Results indicated that activation of AKT phosphorylation by Ca²⁺ restoration following TTFields application is calcium dependent and following calcium restoration AKT phosphorylation (Ser 473) is reactive. Relative protein densities of Akt on the Ser-473 ratio were determined. Each relative protein value was further normalized to the value seen in TTFields application for 72 hours value. Data represents the average of two independent experiments in FIG. 4 and the average of three experiments in FIG. 5.

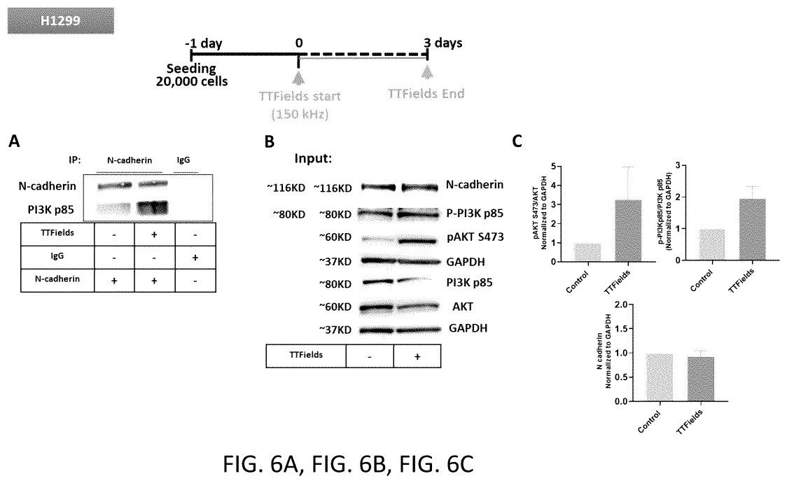

FIG. 6 shows AKT Activation following TTFields is correlated with recruitment of the PI3Kp85 regulatory subunit to N-Cadherin. (FIG. 6A) N-cadherin was immunoprecipitated from H1299 cell lysates with an anti-N-cadherin monoclonal antibody. The presence of the p85 subunit of PI3-kinase in anti-N-cadherin immunoprecipitation were detected by immunoblotting with PI3Kp85 antibody. Untreated cells included as control. (FIG. 6B) Western blot analysis of N-Cadherin, PI3K phosphorylated on P85 subunit, total PI3K, Akt phosphorylated on ser473 and total AKT levels in control and TTFields-treated cells. (FIG. 6C) Quantification of normalized relative levels of P-AKT compared to total AKT levels. normalized relative levels of P-PI3Kp85 compared to total PI3K levels and normalized N-cadherin levels in control and TTFields-treated cells. A value of I was given to the expression level of untreated cells. * p<0.05

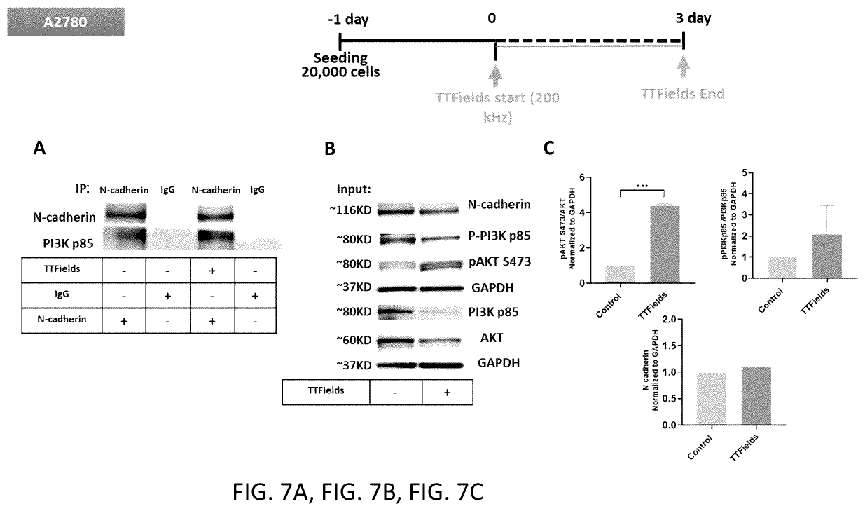

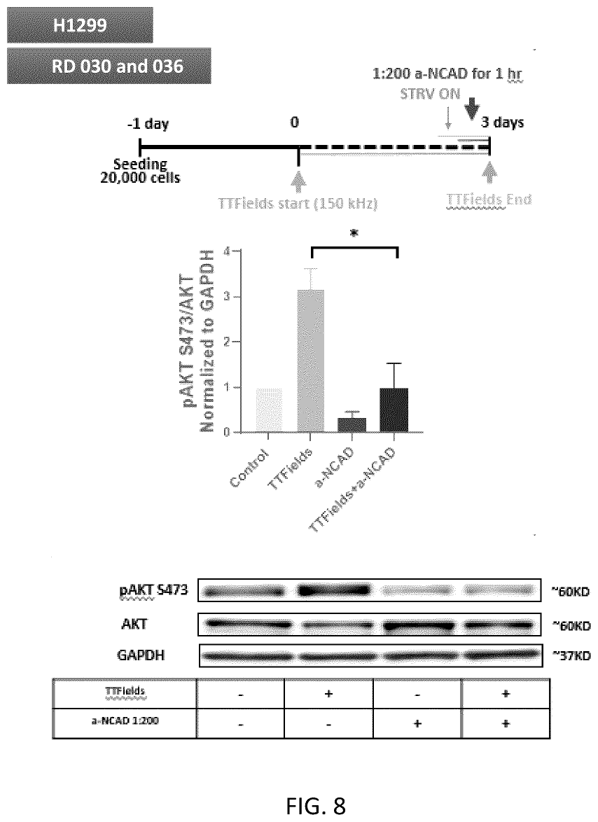

FIG. 7 shows AKT Activation following TTFields is correlated with recruitment of the PI3Kp85 regulatory subunit to N-Cadherin. (FIG. 7A) N-cadherin was immunoprecipitated from A2780 cell lysates with an anti-N-cadherin monoclonal antibody. The presence of the p85 subunit of PI3-kinase in anti-N-cadherin immunoprecipitation were detected by immunoblotting with PI3Kp85 antibody. Untreated cells included as control. (FIG. 7B) Western blot analysis of N-Cadherin, PI3K phosphorylated on P85 subunit, total PI3K, Akt phosphorylated on ser473 and total AKT levels in control and TTFields-treated cells. (FIG. 7C) Quantification of normalized relative levels of P-AKT compared to total AKT levels, normalized relative levels of P-PI3Kp85 compared to total PI3K levels and normalized N-cadherin levels in control and TTFields-treated cells. A value of 1 was given to the expression level of untreated cells. *p<0.05

### B. Example 2

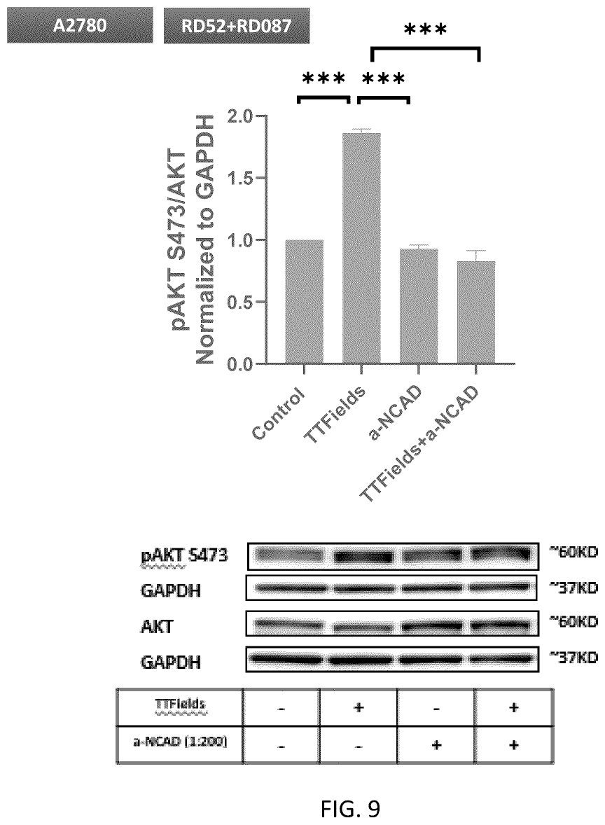

To verify that the activation of AKT was specifically attributed to N-cadherin's homophilic engagement, and not to some other calcium-dependent pathway, a neutralizing assay targeting N-cadherin was employed. The results demonstrate that applying an anti-N-cadherin monoclonal antibody, which prevents N-cadherin from forming cell-cell contacts, to TTFields-treated cells (3 days) resulted in a significant reduction in AKT phosphorylation in both cell lines (H1299 and A2780) (FIGS. 8 and 9).

N-cadherin neutralization assay: A2780 and H1299 cells (2×10⁴ cells/cover slip) were treated with TTFields for 3 days, with serum deprivation for the last treatment day. At treatment end the cells were incubated for 1 hour in the absence or presence of monoclonal anti-N-cadherin antibody (Sigma, C3865, clone GC4; 1:200). Then the cells were lysed and subjected to Western blot analyses to determine AKT phosphorylation, as described above.

### C. Example 3

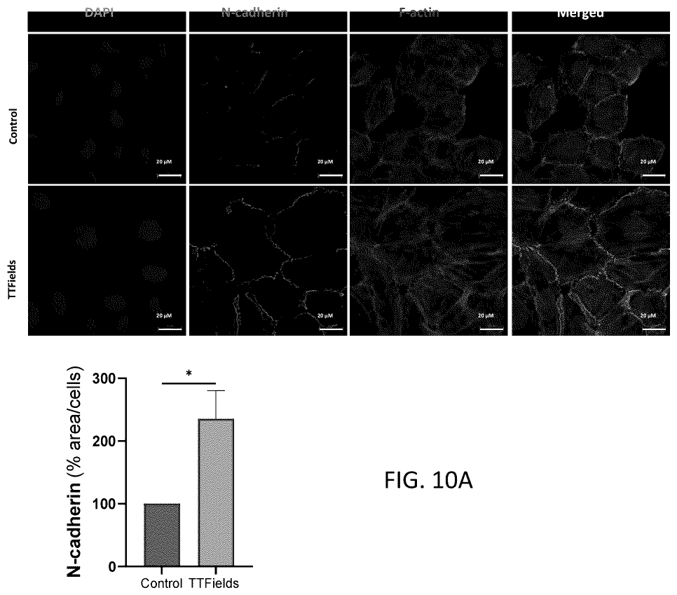

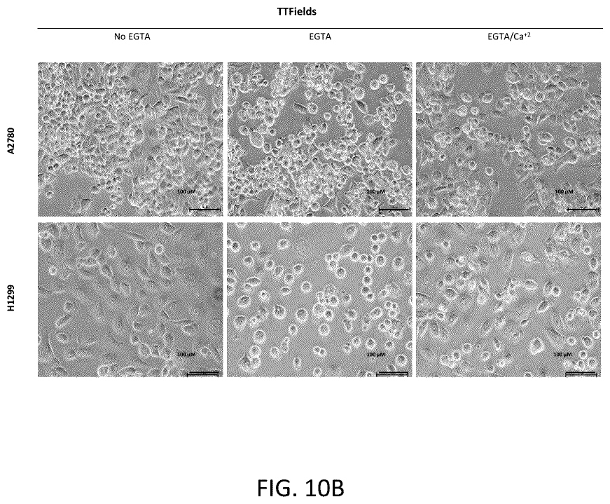

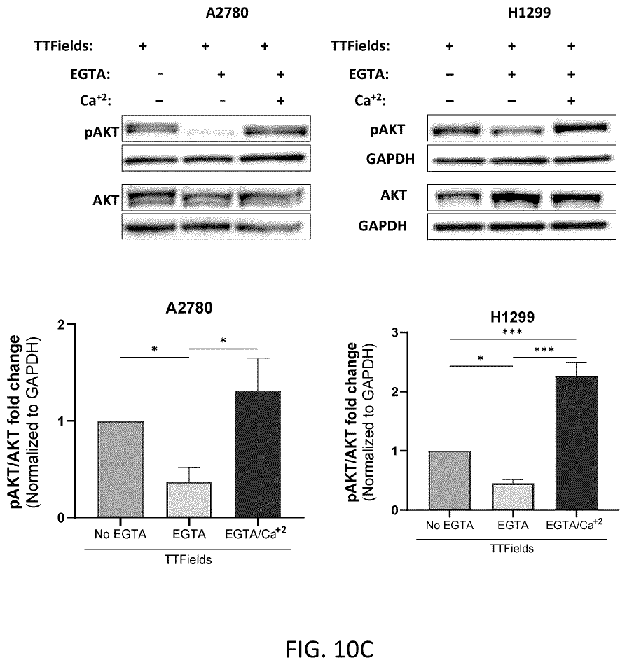

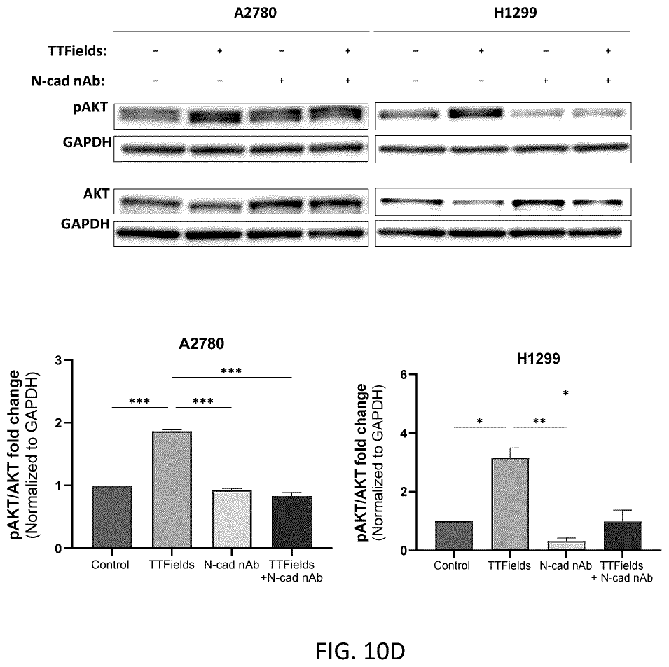

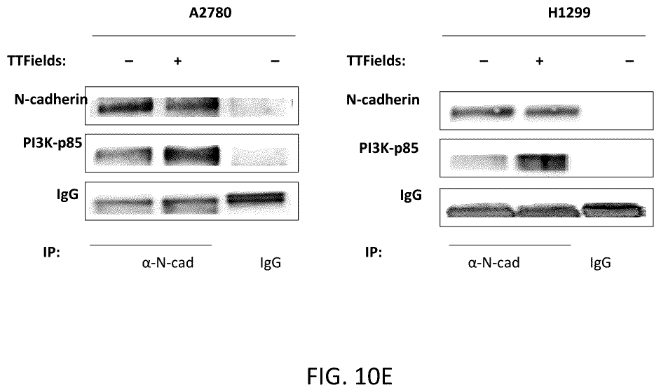

Cell-cell contacts are implicated in rapid activation of the PI3K/AKT pathway through cadherin engagement. The observation that the AKT signal amplitude increased over time during TTFields application prompted the exploration of whether cadherins could affect the intracellular cascades regulating PI3K/AKT activation during treatment. Confocal microscopy of untreated or 72 h TTFields-treated H1299 cells revealed colocalization of N-cadherin and F-actin at cell-cell interfaces, with significantly increased levels of N-cadherin on the membrane of cells treated with TTFields relative to control cells (FIG. 10A).

To determine whether the recruitment of PI3K at sites of N-cadherin homophilic ligation is essential for PI3K/AKT activation in response to TTFields, a calcium switch assay was performed in A2780 and H1299 cells. Epithelial cells require Ca+2 to establish cadherin homophilic ligation; therefore. Ca+2 ions were eliminated from the culture media by addition of a chelating agent (EGTA), followed by reintroduction of Ca+2 ions for restoration of cell-cell contacts. Under both control and TTFields (72 h) conditions, EGTA-treated cells displayed a rounded morphology, with a slight reversal of this phenotype upon calcium restoration (FIG. 10B). Furthermore, calcium elimination resulted in reduced phosphorylation of AKT compared with TTFields-treated cells under normal calcium levels. Engagement of cell-cell adhesion by calcium restoration re-induced AKT phosphorylation (FIG. 10C).

An N-cadherin neutralizing antibody was then used to confirm that AKT activation was a result of N-cadherin homophilic ligation and not perturbations in calcium homeostasis. The results demonstrated that applying the neutralizing antibody, which inhibits N-cadherin-mediated cell-cell contacts, resulted in a significant reduction in AKT phosphorylation as compared with TTFields-treated cells under normal conditions (FIG. 10D).

Next, the recruitment of the p85 regulatory subunit of PI3K to N-cadherin complexes was tested by pull-down assays using an antibody against N-cadherin. In these experiments, it was not distinguished between membranal and cytosolic N-cadherin; accordingly, comparable levels of N-cadherin were found in control and TTFields treated cell lysates (FIG. 10E). In TTFields-treated cell lysates, but not in control lysates, the PI3K-p85 subunit was found to be associated with N-cadherin, indicating increased recruitment of PI3K to N-cadherin complexes following TTFields application. The negative control using a non-specific antibody showed no precipitation of N-cadherin or PI3K-p85. These findings indicate that N-cadherin-mediated cell-cell contacts initiate PI3K-dependent signal transduction, leading to FAK-independent activation of AKT during longer TTFields exposure.

Those skilled in the art will recognize, or be able to ascertain using no more than routine experimentation, many equivalents to the specific embodiments of the method and compositions described herein. Such equivalents are intended to be encompassed by the following claims.

### REFERENCES

1. Shirayoshi et al. “Cadherin cell adhesion molecules with distinct binding specificities share a common structure.” The EMBO Journal, vol 5, issue 10, Oct 1986
2. Cao et al. “Aberrant N-cadherin expression in cancer,” Biomedicine & Pharmacotherapy, vol 118, 109320, Oct. 2019
3. Blaschuk “N-cadherin antagonists as oncology therapeutics” Phil Trans R Soc Lond B Biol Sci. 2015 Feb. 5: 370 (1661): 20140039
4. Mrozik et al. “LCRF-0006, a small molecule mimetic of the N-cadherin antagonist peptide ADH-1, synergistically increases multiple myeloma response to bortezomib,” FASEB Bioadv. 2020 Jun 15: 2 (6): 339-353
5. Gerhardt et al., “N-cadherin mediates pericytic-endothelial interaction during brain angiogenesis in the chicken,” Dev Dyn. 2000 Jul: 218 (3): 472-9
6. Tanaka et al. “Monoclonal antibody targeting of N-cadherin inhibits prostate cancer growth, metastasis and castration resistance,” Nature Medicine volume 16, pages 1414-1420 (2010)
7. El-Kenawi et al. “Angiogenesis inhibitors in cancer therapy: mechanistic perspective on classification and treatment rationales,” Br J Pharmacol. 2013 Oct; 170 (4): 712-729
8. Hamidi et al. “Every step of the way: integrins in cancer progression and metastasis,” Nature Reviews Cancer, volume 18, pages 533-548 (2018)
9. Tran et al. “Signal transduction from N-cadherin increases Bcl-2. Regulation of the phosphatidylinositol 3-kinase/Akt pathway by homophilic adhesion and actin cytoskeletal organization,” J Biol Chem. 2002 Sep 6;277 (36): 32905-14
10. De Santis et al, 2009; Oncogene 28, 1206-1217
11. Li et al, 2001; Cancer Research 61, 3819-3825
12. Pece et al, 1999; J Biol Chem 274:19347-19351.
13. Yamauchi et al, 2011 Cancer Research 71 (14); 2989-97.
14. Volberg et al, 1986, The J of Cell Biology, vol 102, 1832-1842

We claim:

1. A method of treating a subject in need thereof comprising:
a) applying an alternating electric field, at a frequency for a period of time, to a target site of the subject in need thereof; and
b) administering a neural-cadherin (N-cadherin) inhibitor to the subject in need thereof.

2. The method of claim 1, wherein the N-cadherin inhibitor is a calcium chelator or a N-cadherin antagonist.

3. The method of claim 2, wherein the calcium chelator is EGTA, Edetic acid, Citric acid, Edetate disodium anhydrous, or Edetate calcium disodium anhydrous.

4. The method of claim 2, wherein the N-cadherin antagonist is LCRF-0006, ADH-1, HAV-containing peptide, Trp-containing peptide, Compound 15, HAV dimeric, HAV-biomaterial, N-cadherin antibody, H-SWTLYTPSGQSK-NH2.

5. The method of claim 4, wherein the N-cadherin antibody is GC4, 2A9, or 1H7.

6. The method of claim 1, wherein the subject has cancer.

7. The method of claim 6, wherein the cancer is ovarian cancer or non-small cell lung cancer.

8. The method of claim 1, wherein the N-cadherin inhibitor reduces AKT phosphorylation, reduces PI3K/p85 recruitment to N-cadherin, or reduces metastasis in the subject.

9. A method of reducing AKT phosphorylation or inhibiting the recruitment of the p85 subunit of PI3K to an N-cadherin complex in response to alternating electric fields comprising:
a) applying an alternating electric field, at a frequency for a period of time, to a population of cells comprising one or more fibroblasts; and
b) contacting a calcium chelator to the population of cells.

10. A method of preventing metastasis in a subject having cancer comprising:
a) applying an alternating electric field, at a frequency for a period of time, to a population of cells comprising one or more cancer cells; and
b) contacting a N-cadherin inhibitor to the population of cells.

11. The method of claim 10, wherein the N-cadherin inhibitor is a calcium chelator or an N-cadherin antagonist.

12. The method of claim 1, wherein the alternating electric field is applied before, after, or simultaneously with administering the N-cadherin inhibitor to the subject.

13. The method of claim 1, wherein the frequency of the alternating electric field is between 50 kHz and 1 MHz.

14. The method of claim 1, wherein the frequency of the alternating electric field is about 150 or 250 kHz.

15. The method of claim 1, wherein the alternating electric field has a field strength of between 0.5 and 10 V/cm RMS.

16. The method of claim 1, further comprising administering a cancer therapeutic.

17. The method of claim 1, wherein after step a) and prior to step b) detecting an increase in N-cadherin expression in the subject.

18. The method of claim 1, wherein step b) is performed 1, 2, 3, 4, 5, 6, 7, 8, 9, or 10 days after step a) is performed.

19. The method of claim 1, wherein the target site comprises cancer cells.

20. The method of claim 19, wherein the cancer cells are ovarian cancer cells or non-small cell lung cancer cells.


SEQUENCE LISTING (4 sequences)

SEQ ID NO: 1 - protein, 5 aa, synthetic construct
CHAVC

SEQ ID NO: 2 - protein, 9 aa, synthetic construct
LRAHAVDNG

SEQ ID NO: 3 - protein, 12 aa, synthetic construct
SWTLYTPSGQSK

SEQ ID NO: 4 - protein, 14 aa, synthetic construct
CHAVDINGHAVDIC